(19) United States
(12) Patent Application Publication
(10) Pub. No.: US 2024/0032900 A1
(43) Pub. Date: Feb. 1, 2024

(54) CASING FOR CAPTURING AND ANALYSING ANIMAL URINE, DEVICE AND METHOD IMPLEMENTING SAME

(71) Applicant: NOVANDSAT, MURLES (FR)

(72) Inventor: Philippe DAURENJOU, Murles (FR)

(21) Appl. No.: 17/754,492

(22) PCT Filed: Oct. 3, 2019
(86) PCT No.: PCT/FR2019/052352

§ 371 Date: Apr. 4, 2022

(30) Foreign Application Priority Data
Oct. 3, 2018 (FR) 1859166

(51) Int. Cl. A61B 10/00; A01K 1/01; B01L 3/00; G01N 33/52

(52) U.S. Cl. CPC A61B 10/007; A01K 1/0107; B01L 3/5023; G01N 33/528; B01L 2200/16; B01L 2300/0825; B01L 2400/0633; B01L 2300/069; A61B 2503/40

(57) ABSTRACT

The casing for capturing and analysing animal urine comprises a first urine receptacle (188) and a second urine receptacle (191) which is provided with a support (187) for a strip (195) which carries test specimens comprising reagents, the receptacles being separated by a partition wall (156) which is provided with a passage (157), and at least one support which forms a tilting axis between:
a first configuration in which, when the passage is open, the urine flows as a result of gravity into the first receptacle and
a second configuration in which, when the passage is open, the urine flows as a result of gravity into the second receptacle.

### TECHNICAL FIELD OF THE INVENTION

The present invention relates to a casing for capturing and analyzing animal urine, a device and a method implementing this casing.

It applies, among others, to the veterinary field and, more specifically, to monitoring the health of cats and dogs.

### STATE OF THE ART

Veterinarians use urine strips to analyze the urine of animals. However, since appointments are spread over the workday, the animal generally does not have an empty stomach, has already made physical efforts and may have drunk. The quality of the urine, in physicochemical terms, is therefore variable and a source of diagnosis errors. The stress of the animal brought to the veterinarian also modifies the actual concentrations of some analytes.

Devices are known for owners to collect animal urine, comprising tubes that the user must position under the animal while it urinates. These devices require the animal to be watched so as to be present when it urinates. In addition, these tubes trouble the animal and can encourage them to retain their urine.

### SUBJECT OF THE INVENTION

The present invention aims to remedy all or part of these drawbacks.

To this end, according to a first aspect, the present invention relates to a casing for capturing and analyzing animal urine, which comprises a first urine receptacle and a second urine receptacle, provided with a support for a strip that carries test specimens comprising reagents, said receptacles being separated by a partition wall which is provided with a passage, and at least one support that forms a tilting axis between:
a first configuration in which, when the passage is open, the urine flows as a result of gravity into the first receptacle and
a second configuration in which, when the passage is open, the urine flows as a result of gravity into the second receptacle.

Thanks to these provisions, the casing first collects the urine and then, after being tilted, the casing soaks a strip with the urine collected. The soak period is therefore controlled, which substantially improves the quality of the analyses. The casing can be transported to a veterinarian and the strip can be analyzed by the animal's owner, for example by comparison with control colors identifying the quantities of components in the residual urine.

In some embodiments:
the casing comprises a valve that shuts or opens the passage between said receptacles;
the valve is a multiport valve that additionally shuts or opens an inlet for urine in the casing;
the valve comprises a holding tank for the first jet of urine entering the casing;
the tank holding the first jet of urine is topped by a partition wall limiting the flow of the urine to the second receptacle, in the second configuration of the casing;
the first receptacle comprises at least one side compartment and a partition wall holding urine in a side compartment when the casing tilts into the second configuration;
the partition wall holding urine in the side compartment has a chicane or an overflow level;
the casing comprises a cover at least opposite the support for a strip, the cover being mobile with respect to a base comprising the receptacles and/or
the cover bears markers for detecting the strip in an image of the cover.

According to a second aspect, the present invention envisages a device for collecting the urine of a predefined animal, which comprises:
a casing for capturing and analyzing urine that is the subject of the invention;
a tray receiving the urinating animal and holding elements inert to the urine, on a screen configured to retain the inert elements and let the urine pass;
a sloping surface, under the screen, for collecting the urine in a convergence area leading to an inlet of the casing for capturing and analyzing urine.

Thanks to these provisions, the animal feels it is on a litter tray when it urinates in the holding tray. In addition, the urine collected comes from at least one jet subsequent to the first jet, which is frequently soiled at the level of the urethra channel and collection support. The urine is therefore more revealing of any health defects of the animal. Lastly, the device is easy to clean because it is only necessary to pass water over the same path as the urine takes, i.e. from the inert elements, on the screen, on the slope and on the capture means and the collection means. A pet owner can therefore carry out a urine collection without the help of a veterinarian. And this collection can be of a better quality than that made by a veterinarian, since the animal can have an empty stomach, not made physical efforts, not have drunk and not be stressed.

In some embodiments, the means for capturing a first jet of urine comprises a tank below the convergence area.

Thanks to these provisions, gravity feeds the first jet of urine into the tank, the urine being collected in the convergence area.

In some embodiments, the means for capturing a first jet of urine comprises a valve on the passage of the urine downstream from the convergence area, the valve being controlled to change position and direct the urine towards a collection area after a certain amount of urine has passed through the valve.

Thanks to these provisions, the collection area can be separate from the convergence area.

In some embodiments, the means for capturing a first jet of urine comprises an absorbent material configured to absorb the urine.

Thanks to these provisions, the urine of the first jet is absorbed and mechanically held in the absorbent material. The risks of this urine spilling, during the operation of the device, are therefore reduced.

In some embodiments, the means for capturing a first jet of urine comprises a chicane.

Thanks to these provisions, the urine of the first jet is retained and prevented from returning into the convergence area.

In some embodiments, the sloping surface comprises, at least for the surface in contact with the urine, a hydrophobic material.

Thanks to these provisions, the flow of the urine is facilitated and the convergence speed of the urine is more stable, which favors the arrival first, in the convergence area, of the urine of the first jet.

In some embodiments, the hydrophobic sloping surface comprises HDPE grade 6.

In some embodiments, the sloping surface comprises two slopes converging towards an intersection line inclined towards the convergence area.

Thanks to these provisions, the shape of the sloping surface favors its cleaning and the regularity of the flow speed of the urine.

In some embodiments, the slope of the sloping surface comprises between three and five angular degrees.

In some embodiments, the sloping surface has an accordion shape whose lower portions slope towards a collection surface inclined towards the convergence area.

Thanks to each of these provisions, the flow speed of the urine is regular.

In some embodiments, the inert elements comprise hydrophobic cylindrical or spherical plastic granules.

Thanks to these provisions, the urine is held weakly by the inert elements and washing them is made easier.

In some embodiments, the urine collection device comprises a removable cover to enclose the inert elements in the holding tray.

Thanks to these provisions, the cleaning of the inert elements is made easier because they are unlikely to escape under the effect of a jet of water.

In some embodiments, the device comprises two trays of identical height, one for holding inert elements, the other for holding cat litter.

Thanks to these provisions, when the respective positions of the trays are switched, one above the other, the animal still has the same height to cross to reach the cat litter or the inert elements. It is therefore not surprised, and the risk of it not urinating is thereby reduced.

In some embodiments, the two trays each comprise interior frames for supporting the other tray. Thanks to these provisions, switching the respective positions of the trays, one above the other, is made easier.

In some embodiments, the device comprises a means for measuring the weight of the predefined animal and/or of the urine collected before sampling.

Thanks to these provisions, the device obtains at least one measurement indicative of the animal's state of health. The weight measurement means also provides information about the animal's behavior (frequency of passage and time on the tray).

In some embodiments, the device comprises a means for measuring the temperature of the animal and/or the urine collected.

Thanks to these provisions, the device obtains at least one measurement indicative of the animal's state of health.

In some embodiments, the device comprises a means for counting the number of passages during a predefined period and/or the duration of the animal's passage in the device.

Thanks to these provisions, intestinal or urinary problems of the animal can be revealed.

In some embodiments, the casing, also called “receptacle”, is transparent and comprises a tank for collected urine and an access plug for access to the tank by a strip which carries test specimens comprising reagents.

Thanks to these provisions, a measurement of the color and clarity of the urine can be made through the receptacle and a strip can be introduced directly into the tank, for example by a veterinarian wanting to perform an additional urine analysis.

In some embodiments, the device comprises a valve in the convergence area, and the receptacle comprises an end-fitting configured to cause the valve to open and allow the urine to flow as a result of gravity into the receptacle.

In some embodiments, the device comprises a valve in the convergence area, and the receptacle comprises an end-fitting configured to cause the valve to open and a means for placing the receptacle under negative pressure to pump the residual urine towards the receptacle.

Thanks to each of these provisions, the convergence area holds the residual urine until a portion of the residual urine is extracted towards the receptacle.

In some embodiments, the receptacle is, at least partially, transparent in the visible spectrum.

Thanks to these provisions, the physical characteristics of the contents of the receptacle can be observed, in particular the color of the urine and the turbidity of the urine.

In some embodiments, the device also comprises a means for analyzing the residual urine collected by the collection means.

Thanks to these provisions, the animal's owner can perform an initial analysis of the urine and, depending on the results of this analysis, decide whether to consult a veterinarian.

In some embodiments, the analysis means comprises a light sensor for capturing the color of at least one test specimen comprising at least one reagent.

Thanks to these provisions, the analysis means can evaluate at least one component quantity in the residual urine collected.

In some embodiments, the light sensor comprises a matrix image sensor and an optic for taking at least one image of a strip carrying a plurality of test specimens.

Thanks to these provisions, the quantities of several components in the urine collected can be evaluated simultaneously.

In some embodiments, the analysis means comprises a means for compensating for variations in the lighting of each test specimen.

Thanks to these provisions, regardless of the variations of lighting, natural or artificial, of the test specimens, the color of each test specimen is evaluated after compensating for these variations.

In some embodiments, the analysis means comprises a means for evaluating the color of the urine collected.

In some embodiments, the analysis means comprises a means for evaluating the clarity of the urine collected.

Thanks to each of these provisions, physical characteristics of the urine collected are evaluated.

In some embodiments, the analysis means comprises a means for comparing:
at least one evaluated color with at least one color limit value; and/or
at least one evaluated component quantity of the urine with at least one quantity limit value.

Thanks to these provisions, the animal's owner can be automatically notified of a possible anomaly in the composition of the urine and a potential health problem of the animal. The owner can therefore decide whether to consult a veterinarian based on objective elements.

In some embodiments, the analysis means comprises a means for remotely transmitting evaluated colors and/or results of comparing evaluated colors.

In some embodiments, the analysis means comprises a means for receiving from a remote transmitter limit values of color and/or component quantities in the urine.

Thanks to each of these provisions, the practitioner or the veterinarian can receive and/or parameterize the urine analysis performed by the analysis means.

According to a third aspect, the invention envisages a method for using a casing that is the subject of the invention, or a device that is the subject of the invention, which comprises:
a step of capturing an image of the casing;
a step of identifying a strip in the captured image;
a step of determining the color of pads carried by the strip;
a step of comparing at least one pad color with at least one color limit value, for detecting an anomaly;
a step of identifying at least one compartment holding urine, in the captured image;
a step of measuring the turbidity and/or color of the urine held in at least one compartment; and
a step of comparing the turbidity measured and/or the color measured with at least one limit value for detecting an anomaly; and
a step of displaying each anomaly detected.

In some embodiments, the method also comprises:
a step of initializing anomaly detection limit values; and
a step of re-evaluating at least one limit value as a function of values measured for the animal in question.

In some embodiments, the method also comprises:
a first step of determining a behavior of the animal with regard to the casing, for example time, duration and frequency of passage;
a step of initializing limit values relative to the animal's behavior;
a second step of values representative of a behavior of the animal; and
a step of comparing measurements with the limit values, for detecting for an anomaly.

In some embodiments, the method also comprises:
a first step of determining the weight of the animal and/or the weight of at least one urination of the animal;
a step of initializing limit values relative to the weight determined;
a second step of measuring the weight of the animal and/or the weight of at least one urination of the animal; and
a step of comparing measurements with the limit values, for detecting for an anomaly.

As the particular features, advantages and aims of this method are similar to those of the casing and/or the device that is the subject of the invention, they are not repeated here.

### BRIEF DESCRIPTION OF THE FIGURES

Other advantages, aims and features of the present invention will become apparent from the description that will follow, made, as a non-limiting example, with reference to the drawings included in an appendix, in which:

FIG. 1 is a cross-section view of a first particular embodiment of the device that is the subject of the invention;

FIG. 2 is a top view of the device illustrated in FIG. 1;

FIGS. 3 and 4 are cross-section views of the device illustrated in FIGS. 1 and 2;

FIG. 5 is a perspective view of the device illustrated in FIGS. 1 to 4;

FIG. 6 represents, schematically and in cross-section, a first particular embodiment of a means for capturing urine;

FIG. 7 represents, in perspective, a second particular embodiment of a means for capturing urine;

FIG. 8 represents, in perspective and in the interior of the device, a valve opposite a urine convergence area;

FIG. 9 represents, in perspective, a second embodiment of a means for collecting residual urine;

FIG. 10 represents, in a top view, a third embodiment of a means for collecting residual urine;

FIG. 11 represents, in perspective, a fourth embodiment of a means for collecting residual urine;

FIG. 12 represents, according to two views and according to a cross-section, a fifth embodiment of a means for collecting residual urine;

FIG. 13 represents a color chart for comparing colors of test specimens comprising reagents;

FIG. 14 represents, in the form of a logical diagram, steps in a method of operating a device that is the subject of the invention;

FIG. 15 represents, in the form of a block diagram, a means for analyzing a urine sample;

FIG. 16 represents, in the form of a logical diagram, steps of analyzing a urine sample;

FIG. 17 is a perspective view of a second particular embodiment of the device that is the subject of the invention;

FIG. 18 is a top view of a lower portion of the device illustrated in FIG. 17;

FIG. 19 is an elevation view of a base of the device illustrated in FIGS. 17 and 18;

FIG. 20 is a top view of the base illustrated in FIG. 19;

FIGS. 21 to 23 are, respectively, elevation, cross-section and top views of a removable casing for capturing and collecting urine;

FIGS. 24 and 25 are, respectively, top and side views of a bottom of the casing illustrated in FIGS. 21 to 23;

FIG. 26 represents, in a side view, a flange of the casing illustrated in FIGS. 21 to 25; and

FIG. 27 is a top view of a cover of the casing illustrated in FIGS. 21 to 26.

### DESCRIPTION OF EXAMPLES OF REALIZATION OF THE INVENTION

The present description is given in a non-limiting way, in which each characteristic of an embodiment can be combined with any other characteristic of any other embodiment in an advantageous way. It is now noted that the figures are not to scale.

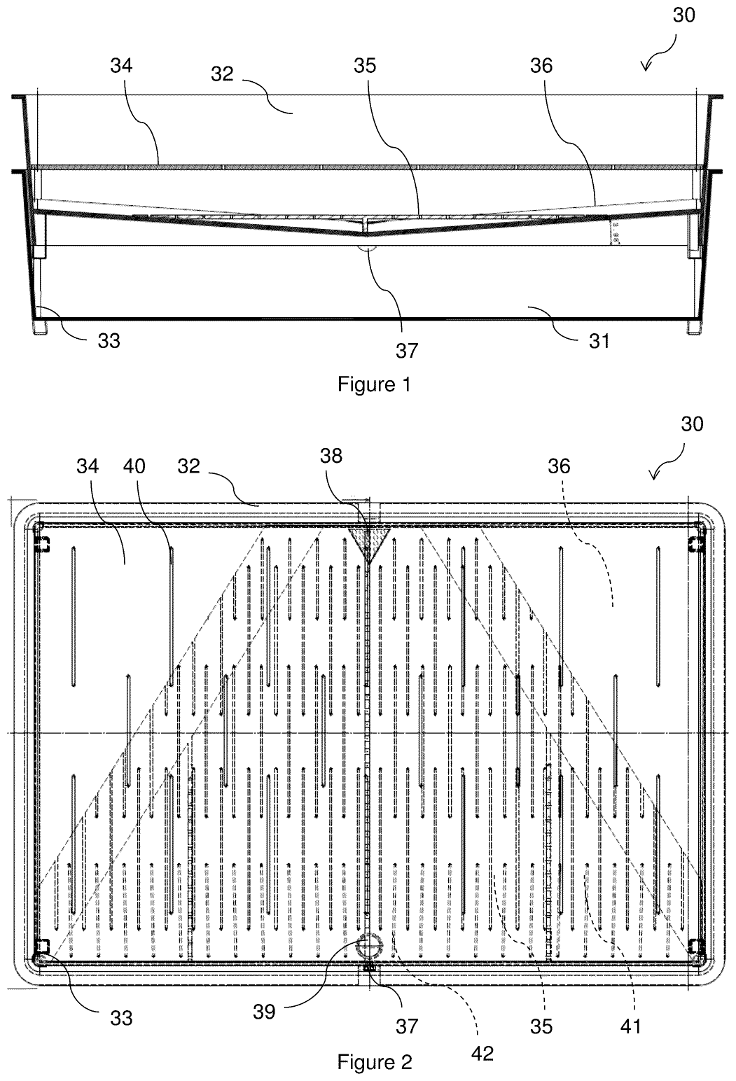

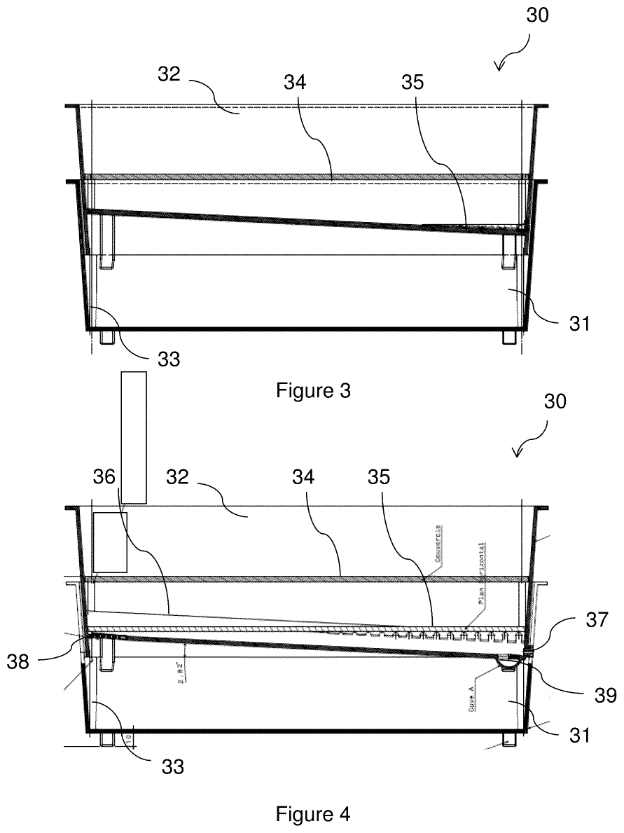

FIGS. 1 to 5 shows a urine collection device 30 that is the subject of the invention, which comprises, in its urine collection configuration, a lower tray 31 and an upper tray 32. The lower tray 31 is intended to receive cat litter so that a predefined animal, in particular a cat or a dog, urinates in it. In the most common configuration, not including urine collection, the respective positions of trays 31 and 32 are switched, with respect to what is shown in FIGS. 1 to 5, so that the animal can mount into tray 31, which is then above tray 32.

The trays 31 and 32 preferably comprise frames 33 forming a support for the other tray, respectively 32 and 31 such that, in the two configurations of superimposition of the trays, the animal has the same height to reach to urinate. This therefore avoids the animal being surprised and dissuaded from mounting into the higher tray to urinate in it.

Tray 32 supports a screen 35 holding elements (not shown) inert to urine and letting the urine pass through slots 41. While the inert elements and the device are being washed, the tray 32 is equipped with a cover 35 letting the water pass through slots 40 whose width does not allow the inert elements to pass.

A sloping surface 36 makes the urine converge towards a convergence area 42 where a means 39 for capturing a predefined volume of first jet urine and a means 37 for collecting a portion of the residual urine are located. Opposite the convergence area, relative to the center of the tray 32, i.e. at the highest point of the intersection lines of the flat sloping surfaces, an outlet 38 is located that makes it possible to manage the overflow of the tray and empty the rinse water.

Therefore, the device 30 for collecting the urine of a predefined animal comprises:
a tray 32 holding elements inert to the urine, on a screen 35 configured to retain the inert elements and let the urine pass;
a sloping surface 36, under the screen 34, for collecting the urine in a convergence area 42;
a means 39 for capturing a predefined volume of first jet urine arriving first in the convergence area 42; and
a means 37 for collecting a portion of the residual urine.

Consequently, the animal feels it is on a litter tray when it urinates in the holding tray. In addition, the urine collected comes from at least one jet subsequent to the first jet. The urine is therefore more revealing of any health defects of the animal. The device 30 is easy to clean because it is only necessary to pass water into the inert elements, on the screen, on the slope and on the capture means 39 and the collection means 37. A pet owner can therefore carry out a urine collection without the help of a veterinarian. And this collection can be of a better quality than that made by a veterinarian, since the animal can have an empty stomach, not made physical efforts and not have drunk.

Preferably, the sloping surface 36 comprises, at least for the surface in contact with the urine, a hydrophobic material. The flow of the urine is therefore facilitated and the convergence speed of the urine is more stable, which favors the arrival first, in the convergence area, of the urine of the first jet. For example, the hydrophobic sloping surface comprises HDPE grade 6.

In the embodiment shown in FIGS. 1 to 5, the sloping surface 36 comprises two slopes converging towards an intersection line inclined towards the convergence area. This shape of the sloping surface favors its cleaning and the regularity of the flow speed of the urine. Preferably, the slope of the sloping surface 36 comprises between three and five angular degrees. This slope means that the first jet urine can arrive first in the convergence area.

In some embodiments not shown, the sloping surface has an accordion shape, a succession of substantially rectangular surfaces forming channels that have “V”-shaped cross-sections, the lower portions of the channels sloping towards a collection surface, perpendicular to the channels, inclined towards the convergence area. The flow speed of the urine is therefore regular and the first jet of urine arrives first in the convergence area.

The inert elements preferably comprise hydrophobic cylindrical or spherical plastic granules. The urine is therefore held weakly by the inert elements and washing them is made easier by their shape and their hydrophobic nature.

For example, the granules are Purell (registered trademark) Grade 6 (medical) non absorbent plastic granules, doc Purell ga7760, or USP class 6 HDPE plastic granules (supplier such as ALBIS, registered trademark). These granules simulate conventional cat litter.

The usable volume of the capture means is adjusted according to the predefined animal species (e.g. dog or cat) to correspond to a volume of a first jet of urine in relation to the total urination volume of the animal and presumed unusable for the analysis. This usable volume of urine is preferably kept trapped in the bottom of the receptacle to be made unusable for the rest of the analysis. The trapping of this usable volume is, for example, a pad of cellulose acetate or other absorbent material, and/or a system of at least one mechanical chicane keeping the volume at the bottom of the capture means. A second supernatant volume, called the “buffer volume”, is between the volume made captive or “settlement volume” at the bottom of the device and the biological liquid usable for analysis, or “flotation volume”.

In the first embodiment illustrated in FIG. 6, the means 45 for capturing first jet urine comprises, below the convergence area 42, a channel 47 equipped with a urine passage sensor 48, e.g. one intended to measure the capacitance between the plates of a capacitor between which the urine passes. The sensor 48 is configured to count the number of urine passage sequences during a predefined period, for example a half-day or a day, and/or sequences to evaluate the urine quantity passing through the channel 47.

A valve 49 on the passage of the urine, downstream from the convergence area 42, is commanded, based on the signal sent by the sensor 48, to pass from a position orienting the urine towards a channel 50 emerging at a tank 52, until a predefined quantity of urine has passed, to a position orienting the urine towards a collection channel 51 emerging at a collection area (not shown). The collection area can therefore be easily separated from the convergence area and the risk of mixing the first discharge urine, on the one hand, with residual urine, on the other hand, is reduced.

Therefore, a urine/air detector 48 signals each passage of urine, which provides a frequency of urine and thus possible cases of cystitis or urinary infections. Optionally, a pump (not shown) directs the first jet of urine towards the waste based on a timescale that will be specific to the typology of the animal (race, age, weight, etc.).

The solenoid valve 49 switches to direct the urine following the first jet into a tube, casing or receptacle for analysis. Once the tube, receptacle or casing is filled, a detector switches the solenoid valve to the waste.

These functions make it possible to have an automatic and completely autonomous device for measuring the passage frequency of the animal and/or measuring the frequency and/or volume of urination (diagnostic interest)

In the second embodiment illustrated in FIG. 7, the means 60 for capturing first jet urine comprises, below the convergence area 42, a tank 61 equipped with a chicane 63 and an overflow channel 62 leading to an outlet for urine 64. When the animal urinates, the first jet of urine flows into the tank 61 and is blocked there by the chicane 63. The chicane 63 holds the first jet urine and prevents it from returning into the convergence area. The residual urine fills the tank 61 until the tank 61 is full and the urine overflows by the overflow channel 62 and reaches the collection area.

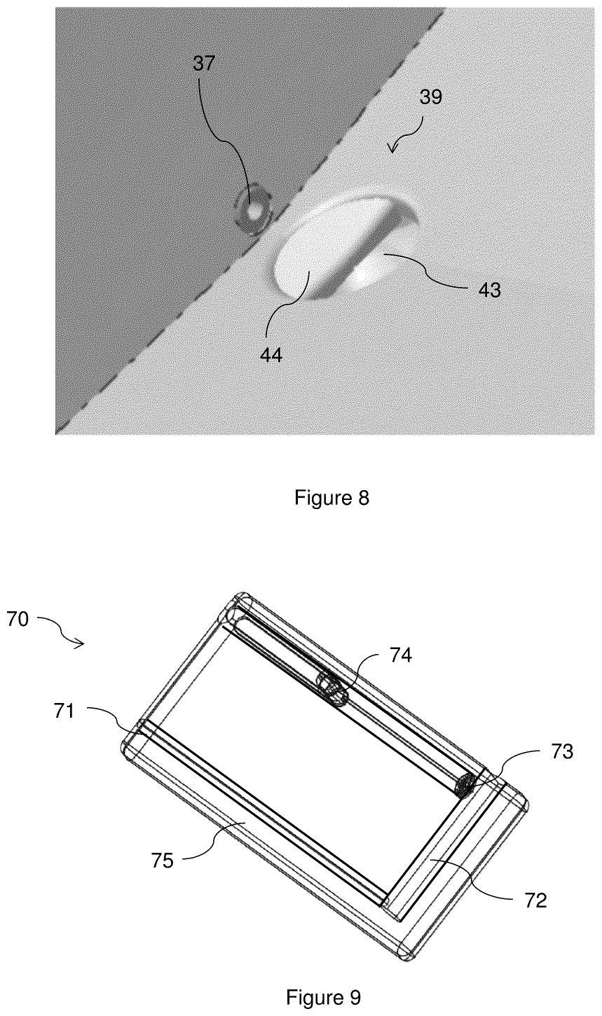

FIG. 8 shows the elements described with reference to FIGS. 1 to 5 and 7, the collection means being formed of a valve 37. It also shows a means 39 for capturing first jet urine, formed of a tank 43 under the convergence area, this tank being equipped with a chicane 44.

The usable volume of the tank is, for example, between 0.5 ml and 2 ml depending on the animal species. The heavy fractions of the first jet of urine settle at the bottom of this tank 43 and the chicane 44 restricts the free flotation of the soiled liquid thus trapped. The lighter fraction representing the second jet is supernatant either in the tray itself or in a tank that will contain the volume required for the collection and the analyses.

In the cases where the urine capture means comprises a tank below the convergence area, gravity feeds the first jet of urine into the tank, the urine collection being carried out in the convergence area or in another tank.

In some embodiments, the means for capturing a first jet of urine comprises an absorbent material (not shown, for example cellulose acetate) configured to absorb the urine. In this way, the urine of the first jet is absorbed and held in the absorbent material. The risks of this urine spilling, during the operation of the device, are therefore reduced.

In some embodiments, the device 30 comprises a means (not shown) for measuring the weight of the animal and/or of the urine collected before sampling. In this way, the device obtains at least one measurement indicative of the animal's state of health. In fact, changes in the animal's weight and changes in the quantity of urine are indicators of some pathologies.

For example, the tray 32 is equipped on its periphery with four mounts allowing weight measurement sensors of type μcell (registered trademark) of 5 kg force to be incorporated. These mounts of the tray 32 incorporating the weight sensors make it possible on a daily basis to:
a. check the presence and frequency of passage of the animal;
b. identify the animal by its weight;
c. evaluate the weight of urine, between the weight measurements before and after the animal's passage;
d. detect any weight gain or loss in the intervals set by the veterinarian.

It is noted that the measurement of the animal's weight and/or of the urine can be performed whether tray 32 is above tray 31 or below it. This information about the frequency, weight gain or loss compared to biological limit values specific to the animal established by the veterinarian makes it possible, if necessary, to perform additional biological analyses, always at home.

In some embodiments, the device 30 comprises a means (not shown) for measuring the temperature of the animal and/or the urine collected. In this way, the device obtains at least one measurement indicative of the animal's state of health. In fact, changes in the animal's temperature are an indicator of some pathologies.

In some embodiments, the device comprises a means (not shown) for counting the number of passages during a predefined period and/or the duration of the animal's passage in the device. In this way, intestinal or urinary problems of the animal can be revealed.

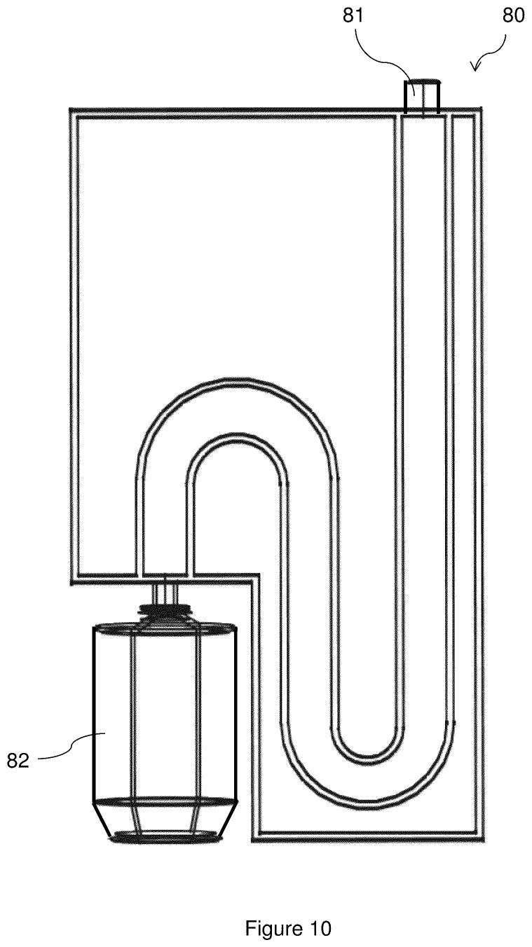

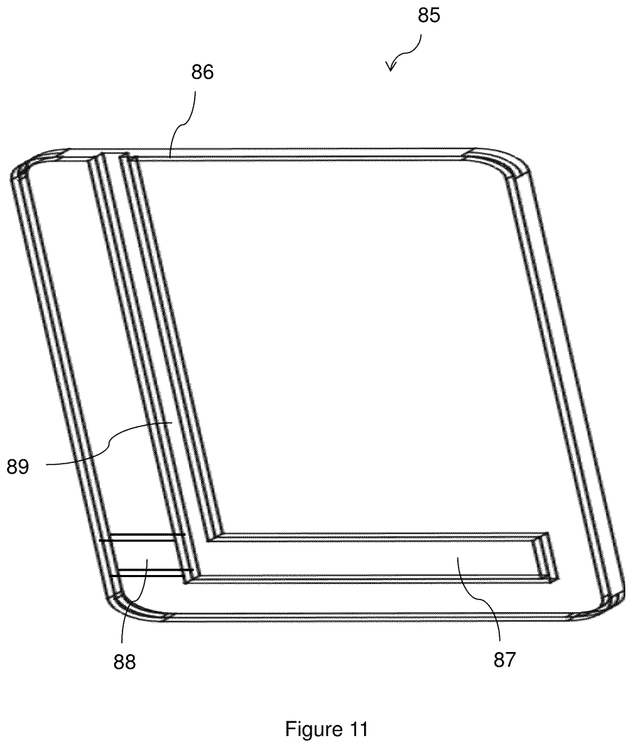

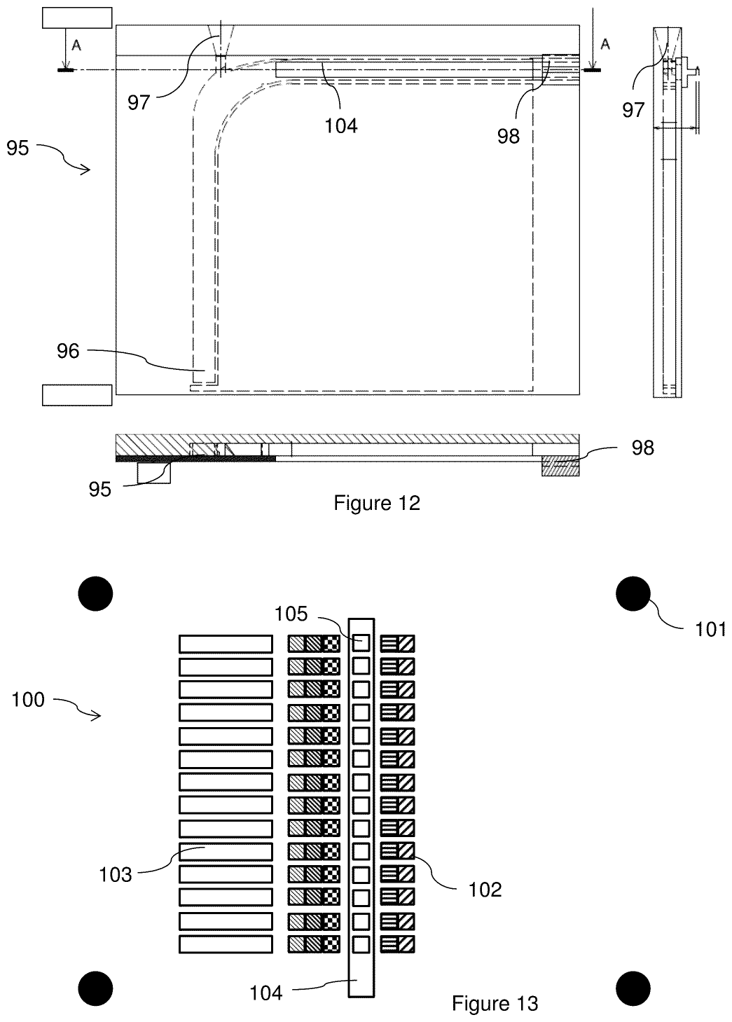

FIGS. 9 to 12 show a casing or receptacle 70 (FIG. 9), 80 (FIG. 10), 85 (FIG. 11) or 90 (FIG. 12) configured to receive a portion of the residual urine coming from the convergence area.

The receptacle 70 comprises an inlet 71 configured to open the valve 37, a channel 75 leading to a tank 72 and a piston 73 put into motion by handles 74 that can be accessed by the user from the outside of the receptacle 70. By pulling on the piston 73, the user pumps a portion of the residual urine present in the convergence area 42.

It can be seen that the receptacle 70 is configured to receive a strip (not shown), which carries test specimens comprising reagents, in the channel 75.

In the variant 80 of the receptacle or casing illustrated in FIG. 10, the inlet 81 has a shape suited to the valve 37 so as to open this valve 37, by pressure of the inlet 81 on the valve 37. The piston 73 is replaced by a bulb 83. Pressing on the bulb 83 before pressing the inlet 81 on the valve 37, and then releasing the bulb 83 once the valve 37 is open, causes the pumping, by negative pressure, of a portion of the residual urine in the convergence area 42.

In the variant 85 of the receptacle or casing illustrated in FIG. 11, the inlet 86 leads to a channel 89 configured to receive a strip (not shown), which carries test specimens comprising reagents. A tank 87 is located at the end of the channel 89.

The receptacle or casing 85 comprises a fitting configured to cause the valve 37 to open, and allow the urine to flow as a result of gravity into the receptacle.

A plug 88 allows a strip to pass directly into the tank 87. This plug is, for example, used by a veterinarian to perform an additional analysis of the urine collected.

FIG. 12 shows a transparent receptacle or casing 90 comprising a right-angle channel to form a tank 96 extended by an inlet 97 and an impregnation area of a strip 104 secured to a marker 98. A cover 95 allows the interior of the receptacle 90 to be cleaned.

The embodiments of the receptacle 85 and 90 fulfills the requirements for:
keeping a sample of urine for possible additional analysis by the veterinarian;
enabling an image of the color and clarity of the urine sample to be acquired;
enabling the strip to be mechanically immersed (simple and repeatable) by linear translation; and
easier cleaning.

The receptacles 70 and 80 comprise a fitting configured to cause the valve to open and a means for placing the receptacle under negative pressure to pump the residual urine towards the receptacle.

The receptacle 70, 80, 85 or 90 can be transported to a veterinarian. The strip can be analyzed by the animal's owner, for example by comparison with control colors identifying the quantities of components in the residual urine (see the color chart 100 illustrated in FIG. 13).

Preferably, the receptacle 70, 80, 85 or 90 is, at least partially, transparent in the visible spectrum. As a result, the physical characteristics of the contents of the receptacle can be observed, in particular the color of the urine and the turbidity or clarity of the urine. A transparent collection receptacle makes it possible to take a photo of the clarity and transparency of the urine sample, useful data for the biological diagnosis.

The inlet 71, 81, 86 or 97 of the receptacle 70, 80, 85 or 90 is preferably equipped with a plug.

In a variant, the receptacle 70, 80 or 85 has an outside shape of an elongated tube, or “pen”.

For a veterinarian, the advantage of the receptacle 70, 80, 85 or 90 is to be able to read a strip, check the color and clarity of a urine, and have a urine sample for additional analyses, without having to take another sample from the animal.

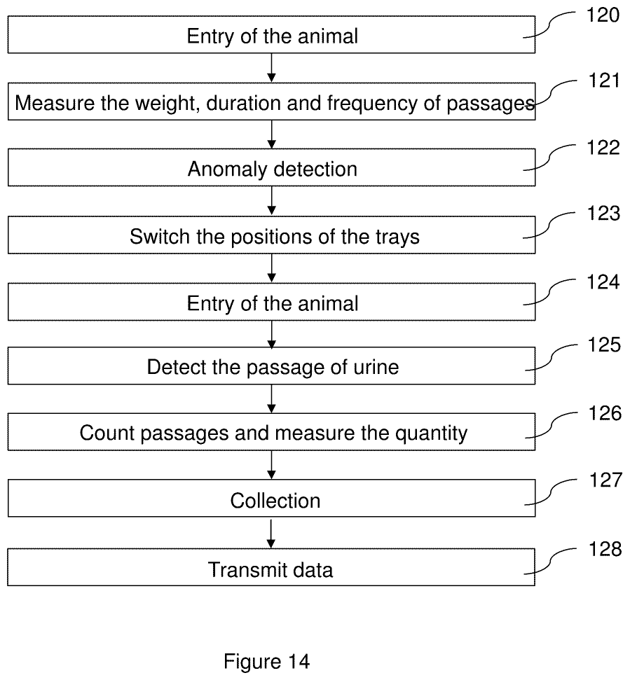

For the utilization of the device that is the subject of the invention, the series of steps illustrated in FIG. 14 can be carried out. At the start of this series of steps, the device is in its configuration where the tray of cat litter 31 is above the urine collection tray 32. During a step 120, the animal mounts on to the tray 32. During a step 121, the device measures the weight of the animal, by the difference between the immediate measurement after stabilization of the animal and the measurement taken after the position of the trays is switched. During this same step 121, the device determines the frequency (the number over a predefined period) and duration of the animal's passage on the tray of cat litter 31. The device records the weight, frequency and duration thus measured.

During the step 122, the device determines a possible anomaly in the data measured, compared to limit values for the animal considered. The device therefore triggers an alert message to the application loaded on the mobile telephone of the animal's owner.

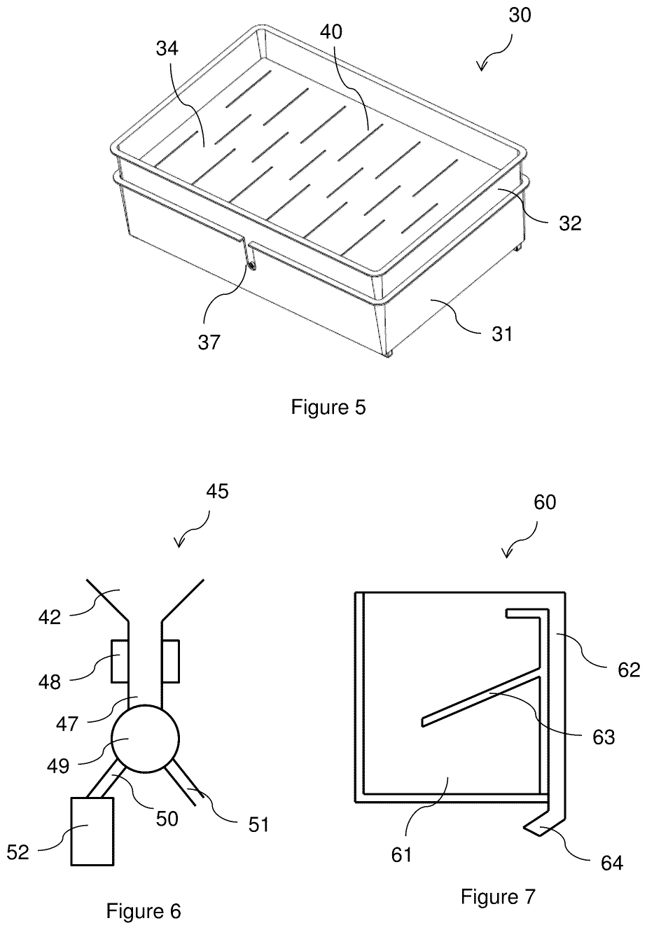

During a step 123, the animal's owner switches the respective positions of trays so that tray 32 is above tray 31, as illustrated in FIGS. 1 and 5, and removes the cover 34. During a step 124, the animal mounts on to the tray 32.

During a step 125, the device detects the passage of urine, for example with the components illustrated in FIG. 6. During a step 126, the device counts the number of times urine has been passed during a predefined period, for example a half-day or day, estimates the quantity of urine (taking into account a quantity remaining held in the granules and on the surfaces in contact with the urine) and records these values. During a step 127, the user takes a sample of residual urine after the first jet urine is captured. During a step 128, the data measured are transmitted, for example to a communicating mobile telephone or to a WiFi box (home internet access gateway). When the owner judges that it is no longer necessary to take urine samples, he places the cover 34 on tray 32, as illustrated in FIGS. 1 to 5 and cleans the tray 32 and the granules it holds by passage of clear water. Then he replaces tray 32 above tray 31.

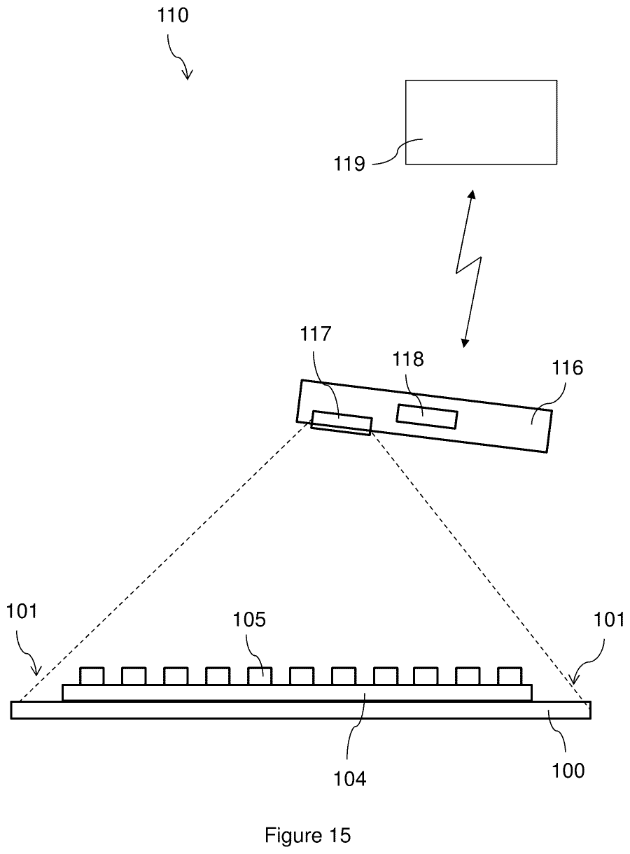

FIG. 15 shows a means 110 for analyzing urine collected, which comprises, on a color chart 100, for example similar to the color chart illustrated in FIG. 13, a strip 104 equipped with a plurality of test specimens 105, each comprising at least one reagent. Each reagent reacts with a specific component of the urine. The change in the color of each test specimen plunged into the collected urine therefore represents a level of the specific component in the urine. The color chart 100 also comprises marks 101 intended to help measure the deformation of the image due to the misalignment of the image sensor and the color chart 100. The color chart 100 shows, opposite each test specimen 105, colors 102 that the test specimens take for predefined levels of components in the urine collected.

With a matrix image sensor 117 equipped with an optic, for example that of a mobile telephone 116, the user takes an image of the strip 104 and the color chart 100. The mobile telephone is configured to communicate with a server 119, via a wireless network, for example a WiFi network or a mobile telephone network. Either a processor 118 of the mobile telephone 116, or the server 119 performs the image processing operations described below to determine the levels of components of the urine that are revealed by the reagents of the test specimens 105.

In this way, with a simple mobile telephone, the animal's owner can perform an initial analysis of the urine to obtain an evaluation of the quantities of several components in the urine collected at the same time and decide whether to consult a veterinarian, based on the results of this analysis.

Preferably, the processor of the mobile telephone 116 or the server 119 is also configured to process an image of the urine present in the receptacle or casing 70, 80, or 90 to determine the color of the urine and also its clarity.

Preferably, the mobile telephone 116 or the server 119 comprises a means for compensating for variations in the lighting of each test specimen. For example, the analysis means 110 comprises a means for comparing the color of each test specimen 105 with the colors 102 of the color chart 100 that are opposite each test specimen. Alternatively, the mobile telephone 116 or the server 119 performs a white balance on the white background of the color chart 100. In this way, regardless of the variations of lighting, natural or artificial, of the test specimens, the color of each test specimen is evaluated after compensating for these variations.

Preferably, the mobile telephone 116 or the server 119 comprises a means for comparing:
at least one evaluated color with at least one color limit value, the color limit values being represented by the colors 102; and/or
at least one evaluated component quantity of the urine with at least one quantity limit value.

The animal's owner can be automatically notified of a possible anomaly in the composition of the urine and a potential health problem of the animal. All these data are shared with the veterinarian, who can therefore decide whether to schedule a consultation for the animal and continue the treatment established. The owner, for his part, can decide whether to consult a veterinarian based on objective elements.

The communication between the mobile telephone 116 and the server 119 comprises the transmission of evaluated colors and/or results of comparing evaluated colors. Preferably, the mobile telephone 116 comprises a means for receiving, from the remote server 119, limit values of color and/or component quantities in the urine.

In this way, the practitioner or the veterinarian can receive and/or parameterize the urine analysis performed by the analysis means.

The remote communication between the analysis means 110 and the server 119 allows the practitioner to carry out:
patient monitoring for comparison specific to the animal (auto-reference);
an enrichment and comparison of a biological database (to improve the detection of symptoms, diagnoses and the effects of treatments).

The different steps required for the extraction from the area of the color chart that corresponds to the color of a test specimen, after acquisition of a digital image, comprise:
searching for marks 101;
tests on the shooting distance between the color chart 100 and the mobile telephone 116;
tests on the perspective, angle between the color chart and the smartphone;
calculating the angle of rotation of the color chart relative to the image sensor 117;
extracting the area of the image where the colors of the color chart are located;
aligning horizontally and vertically by rotation the grid of the color chart;
calculating the perspective deformation of the image; and
correcting perspective defects.

All these steps make it possible to optimize the acquisition of images.

The communication between the mobile telephone 116 and the server 119 comprises an identification of the owner and the animal.

Three different color comparison algorithms can notably be used for recognizing the colors of the pads of a urinary strip compared to a color chart.

These three algorithms are the correlation of histograms in the HSV (acronym for Hue Saturation Value) space, a method of HSV distance measurement, and a method of LAB distance measurement.

The correlation of histograms in the HSV space uses the H and S channels of the image converted to the HSV format. This starts from the principle that the V channel, being tainted by variations of lighting, risks distorting the results. The aim of the algorithm consists of calculating the correlation between the H and S histograms coming from each color 102 of the color chart and the test specimen 105 corresponding to the strip.

After defining the number of classes to be taken into account in each histogram, H and S, the following steps are executed:

for each line of the color chart:
calculating H and S histograms for the test specimen 105 of the strip 104 located on this line;
normalizing these histograms;
   for each reference color 102 of the color chart on this line:
calculating H and S histograms for this reference color;
normalizing these histograms;
calculating the correlation coefficient between the histograms of the reference colors 102 and the color of the test specimen 105.

Then, for each line, the maximum correlation coefficient is searched for, only taken into account if the separation between the largest coefficients is greater than a predefined limit value (relevancy test).

Lastly, a validity test is carried out: the correlation coefficients are considered to be valid if their value exceeds a predefined limit value. In general, in statistics, this limit value is 0.8. However, a limit value as low as 0.1 already indicates a correct recognition of the color.

With regard to the algorithm for measuring the HSV distance, as the HSV space is cylindrical, in order to calculate a Euclidean distance in it, it is necessary to project the H, S, V values over the principal axes. Secondly, the similarity between a reference color and the color of the test specimen is evaluated, by calculating the square of the weighted Euclidean distance that separates them.

A normalization coefficient depends on the weights assigned to each of the H, S and V channels.

Generally, the weight of the V channel can be assigned a very low, even zero, value in order to overcome variations of lighting.

Then, for each line, the maximum similarity is searched for. Lastly, a validity test is carried out: the similarities are considered to be valid if their value exceeds a predefined limit value.

In the case of the algorithm for measuring the LAB distance, as the LAB space is linear by its nature, the weighted Euclidean distance is calculated directly. In a second phase, for each line, the smallest distance is searched for.

The database kept by the server 119 makes it possible to establish a pre-diagnosis of the animal's state of health based on the results obtained by capturing and processing images as well as information about the characteristics of the animal, such as the weight, age, species, race and sex.

The final diagnoses established by the veterinarians are stored in the base, enabling its enrichment as cases are recorded in order to refine the initial pre-diagnoses.

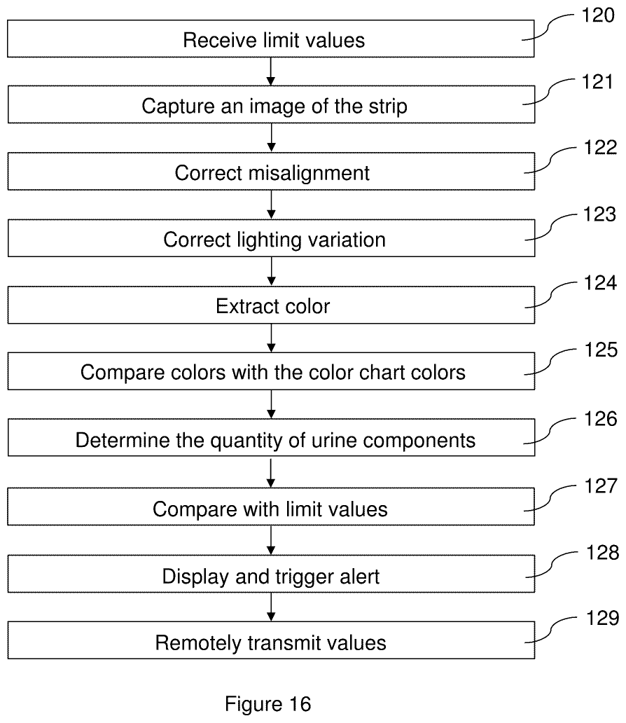

FIG. 16 represents, in the form of a logical diagram, steps of analyzing a urine sample, performed by an application loaded on a mobile telephone 116. During a step 120, the application receives, from the server 119, limit values to be applied for the animal considered. During a step 121, the mobile telephone 116 carries out an image capture of the strip. During a step 122, the application corrects misalignments by applying a deformation to the captured image that renders an image in which the marks 101 form the corners of a rectangle.

During a step 123, the application corrects the variation of lighting. During a step 124, the application extracts the color of each test specimen. During a step 125, the application compares the color of the test specimen with the colors of the color chart that is opposite (i.e. on the same line as) the test specimen considered. It is noted that the steps 123 to 125 can be combined in a single step when correlations, as described above, are performed. During a step 126, the application or the server 119 determines the quantities or levels of components corresponding to the reagents of the test specimens in the urine collected. During a step 127, the application or the server 119 compares the quantity or level values determined during the step 126 with the limit values received during the step 120. During a step 128, the application causes the display, on the screen of the mobile telephone 116, of the results obtained and, if the limit values received are exceeded, an alert message inviting the owner to consult the veterinarian. During a step 129, if the application has performed steps 126 and 127, the application transmits the results obtained to the server 119.

Of course, the distribution of steps between the application and the server 119 can be changed, in variants not described.

FIGS. 17 to 27 represent a second particular embodiment 160 of the device that is the subject of the invention.

As illustrated in FIG. 17, the device 160 comprises an upper half-casing 161 equipped with a handle 159 and a tilting door 162, and a lower half-casing 163 equipped with a base 165, a passage 166 and a drawer 174. The two half-casings 161 and 163 are connected by fasteners 164. When the animal goes to urinate, it enters into the device 160 by pushing the door 162 to make it tilt. When the owner wants to change the cat litter or clean the components of the device, he unlocks the fasteners 164 and lifts the upper half-casing 161 using the handle 159. He therefore has access to the superimposed trays. FIG. 18 illustrates the lower half-casing 163 after the upper half-casing 161 is removed, when the tray of cat litter has been removed or is below the tray holding inert elements. This FIG. 18 shows the tray 167 for receiving the urinating animal and for holding elements inert to the urine, on a screen whose openings 168 in segments of concentric circles are shown. As detailed with regard to the first embodiment of the device, the screen 168 is configured to retain the elements inert to the urine and let the animal's urine pass. A sloping surface (not shown), under the screen 168, collects, as a result of gravity, the urine in a convergence area (not shown), from which the urine flows into a casing 175 for collecting and analyzing urine (see FIGS. 20 to 27).

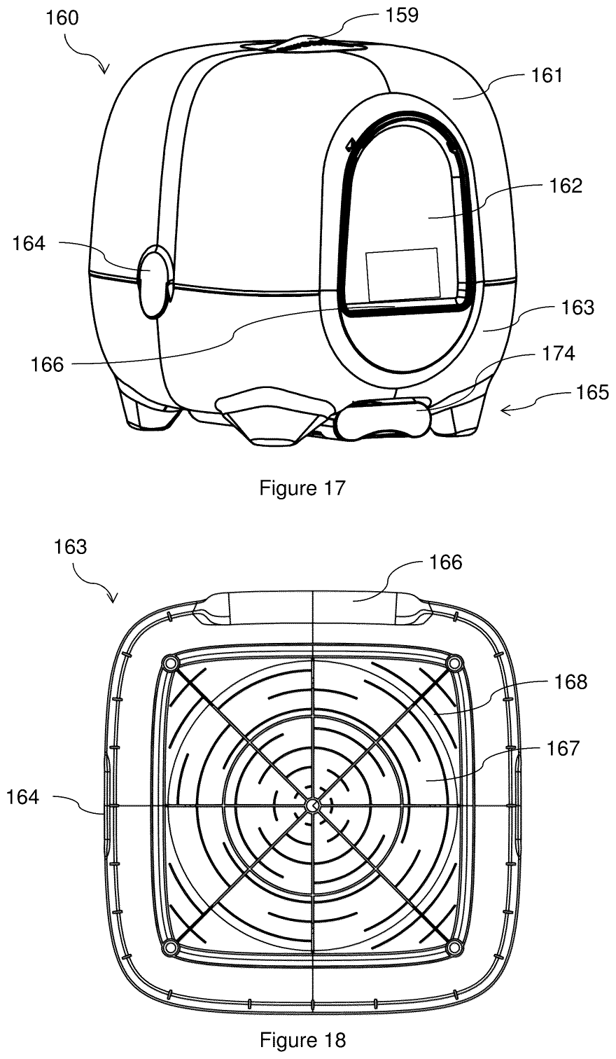

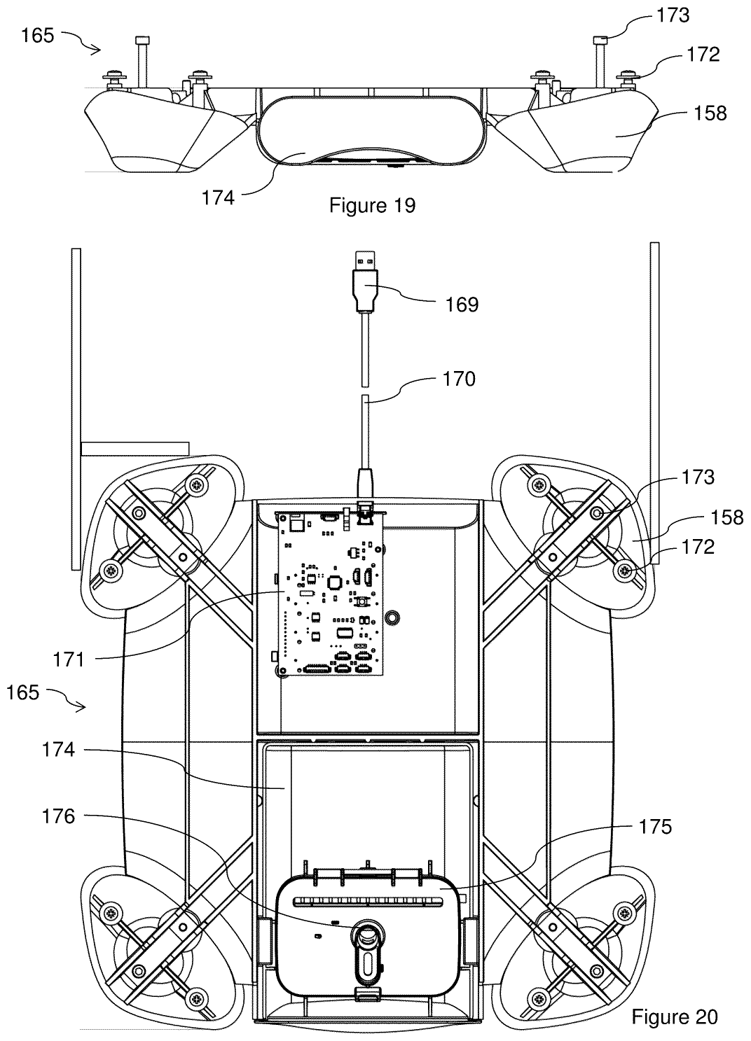

As illustrated in FIGS. 19 and 20, the base 165 comprises four feet 158 equipped with guidance studs 173 and supports 172 for weight measurement. The guidance studs prevent the animal's movements from moving the lower half-casing 163 from the base 165. The supports 172 are collectively subject to the entire weight of the elements of the device, except for the base 175. As detailed above, by comparing the weights measured without the animal, with the animal, and then without the animal but with the urine, the device determines the weight of the animal and its change, and the quantity of urine released by the animal. The base 165 comprises an electronic board 171 equipped with a connector to connect a cable 170 equipped with a second connector 169, for example using the USB specifications (acronym for Universal Serial Bus).

The electronic board 171 comprises a programmable component, for example a microcontroller, and a memory of a data processing program, subsequently also called “algorithm”.

The base also comprises the drawer 174 which retains the removable casing 175 in position. The opening for urine to enter the casing 175 is shown in 176.

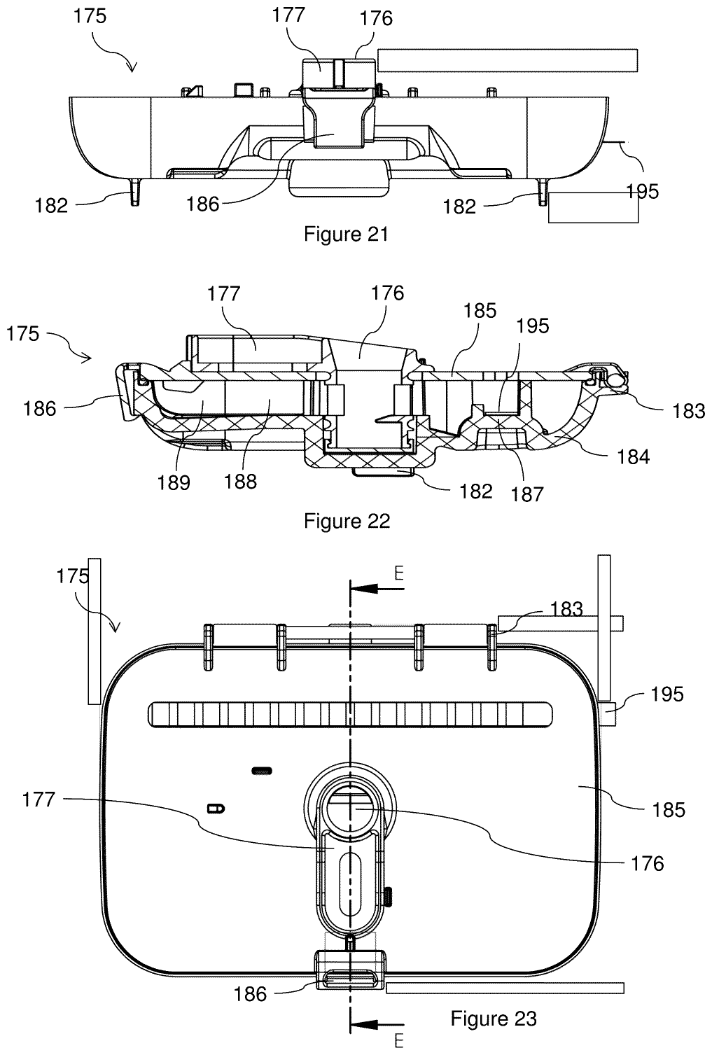

The casing 175 is shown, in greater detail, in FIGS. 21 to 27. The casing 175 has dimensions of approximately 120 mm by 80 mm (top views). The casing keeps a good quality second jet sample, kept at a medical/food grade.

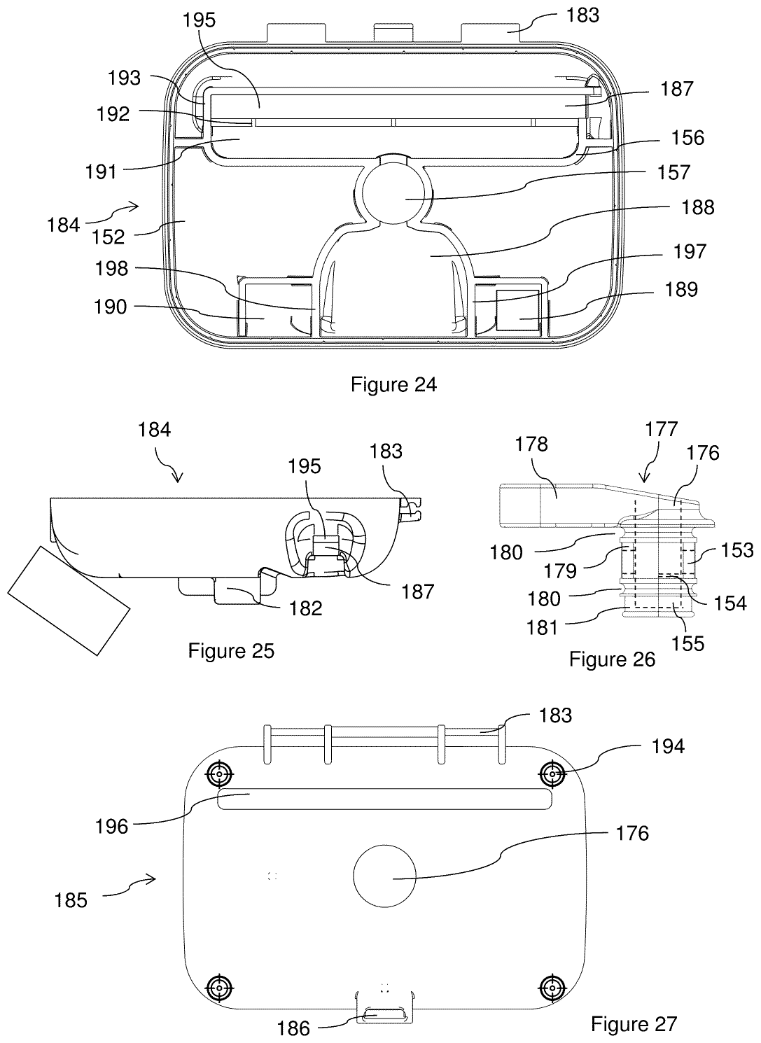

As can be seen in these figures, the casing 175 comprises three elements mobile to each other, a base 184 (see FIGS. 24 and 25), a cover 185 (see FIG. 27) and a flange 177 (see FIG. 26). The cover 185 is mounted on a hinge 183 of the base 184. The cover 185 is locked onto the base 184 by a flexible clip fastener 186. The flange 177 is mobile in rotation around an axis, vertical in FIGS. 21, 22, 25 and 26. The feet 182 form at least one tilting axis of the casing 175. In the embodiment shown in the figures, the feet 182 have an overall rectangular flat shape, the tilting axis passing from one lower apex of the rectangle to another during the tilting. In some variants, the feet have an overall triangular flat shape, of which one apex is oriented towards the bottom and matches the tilting axis. In some variants, the feet have an overall circular flat shape, the tilting axis running along the circle during the tilting.

The base 184 comprises a support 187 for a strip 195, forming a guide in translation. The base 184 comprises a first urine receptacle 188 comprising side compartments 189 and 190. The base 184 also comprises a second receptacle 191 isolated from the first receptacle 188 by a partition wall 156 equipped with a passage 157. The support 187 and guide 195 for the strip comprises a stop 193 and spurs 192.

As shown in FIG. 26, the flange 177 comprises a handle 178, a lower plug 181, two locations of o-rings 180, an interior vertical channel whose upper portion is the urine inlet 176, and a horizontal channel 153. The plug 181 comprises a tank 155 for the first jet urine and a horizontal partition wall 154 holding urine in the tank 181. When the urine enters into the casing 175, by the inlet 176, the casing 175 is in a first configuration in which the urine is directed, as a result of gravity, towards the first receptacle 188. With reference to the intermediate configuration shown in FIG. 22, in the first configuration, the casing 175 has turned counterclockwise and is resting on its feet 182 and on the ridge of the casing located under the first receptacle 188. In this first configuration, the urine begins by filling the first jet urine tank 155, and then the first receptacle. When the animal has finished urinating, the user extracts the casing 175 of the device 160 by opening the drawer 174. Then he tilts the casing 175 into a second configuration in which the urine in the first receptacle 188 that is not held in one of the compartments 189 and 190 passes through the valve 177 to reach the second receptacle 191, as a result of gravity. With reference to the intermediate configuration shown in FIG. 22, in the second configuration, the casing 175 has turned clockwise and is resting on its feet 182 and on the ridge of the casing located under the second receptacle 191. It can be seen that the urine in the tank 155 is held there by the partition wall 154.

In a variant, the tank 155, forming a means for capturing a first jet of urine, comprises an absorbent material configured to absorb the urine, in addition to or in place of the partition wall 154.

After placing the casing 175 in the second configuration, the user closes the multiport valve 177 by making it turn a quarter of a turn relative to its axis. Then, approximately one minute after the impregnation of the strip 195, he captures an image of the cover 185 and, through its windows, of the strip 195 and the urine in the compartments 189 and 190.

The cover 185 is connected to the base 184 by a sealing gasket. The first tank 155 has a volume of the order of 10 to 15 percent of the volume of the urination. The first receptacle 188 has a volume of the order of four milliliters, preferably between three and six milliliters. Each of the compartments 189 and 190 has a volume of the order of one milliliter. The aliquot volume 152 surrounding the first receptacle 188 receives urine by overflow. It has a volume, for example, of 15 to 25 milliliters.

As illustrated in FIG. 27, the cover 185 comprises markers 194 facilitating detection in an image, and a window 196 for observing the strip 195. The cover 185 is transparent to enable the color in the compartment 190 and the turbidity in the compartment 189 to be measured.

The urine in the first receptacle 188 enters into compartments 189 and 190 by overflowing over the partition walls 197 and 198 and/or by passage in the chicanes shown in FIG. 24. The cover preferably has at least one gage of the level of urine in each of the receptacles 188 and 191 and compartments 189 and 190.

All the particular features of one of the embodiments of the casing or receptacle are compatible with those of another embodiment of the casing or receptacle to form variants of these embodiments. All the particular features of one of the embodiments of the device are compatible with those of another embodiment of the device to form variants of these embodiments of the device that is the subject of the invention.

One embodiment of the device that is the subject of the invention is described below. After the acquisition of a device, the veterinarian configures the profile of the animal to establish a first level of limit values (thresholding), customized according to the race, sex, age, weight and medical history of the animal. These first limit values (“thresholds”) concern, in particular, the ideal weight of the animal and the so-called “normal” behavior, and also the concentration levels already recorded, such as the urine glucose level generally recorded in previous tests. After a defined period of the device being used at the animal's home, a second thresholding level is automatically established based on the data collected and processed by the algorithms. These thresholds concern the animal's daily habits, the number of daily passages and the duration of the passages in the device, so as to detect as early as possible any variation in the behavior that can be taken to be the first symptoms of an illness. The limit values (“thresholds”) being exceeded alerts the veterinarian (and/or the owner) so he can decide on the need to perform a urinary test by the casing 175. The results observed from the animal's urinary test enables the veterinarian to refine the control thresholds for the following tests and decide whether the animal needs a medical examination.

During the collection of the urine, the standby position of the casing 175 is inclined in a first configuration in which the urine flows towards the first receptacle 188.

At the same time, the valve 177 is in the configuration shown in FIGS. 21 to 23 allowing the urine to enter the first receptacle.

With regard to the acquisition of the image, the algorithm checks the position of the image sensor (generally of a smartphone, tablet or webcam) in relation to the strip and its support, measures the luminosity, and then takes an image of the entire cover of the casing or open casing (case not detailed below). The shooting settings, which can be varied, are divided into two categories: settings external to the image sensor, and the internal settings. The external controls comprise:
the lighting: uniformity, flickering, intensity, disposition relative to the sensor;
the distance of the shot;
the camera angles: lateral angle and plunge angle; and
the position of the strip in the device.

The internal controls comprise:
the white balance; and
the focusing (autofocus).

With regard to the image processing, the algorithm rectifies the image (angle correction) to be able to identify and partition the various elements that make up the image of the cover. In this way, the various portions and sub-portions essential for data collection are determined. The algorithm identifies the area where the urinary strip is located and then partitions the 15 pads it comprises, thus determining 15 distinct areas (14 “analyte” pads and one reference pad). The 14 analytes concern, for example, the leukocytes, ketone, nitrite, urobilinogen, bilirubin, glucose, proteins, density, hydrogen potential (pH), the presence of blood, ascorbic acid, microalbumin, calcium and creatinine. Two ratios are calculated: UPCR (Urinary Protein/Creatinine) and UMCR (Urinary Microalbumin/Creatinine).

The algorithm also identifies the area of the urine compartments and partitions it into sub-portions: compartment 188 (“Usable” volume), compartment 190 (Color of the urine) and compartment 189 (Turbidity of the urine). The algorithm then analyses the different sub-portions of the image to convert these images into digital data. The colors of the strip's pads are then compared with previously digitized reference color charts to identify the associated digital values.

Three different color comparison algorithms have been selected. These three algorithms are the correlation of histograms in the HSV (acronym for Hue Saturation Value) space, a method of HSV distance measurement, and a method of LAB distance measurement. The algorithm performs a second level of check for the level of light on the reference pad, and adjusts the luminosity variable accordingly to avoid false positives. The color of the urine is determined by comparison with a chart of urine colors, following the same methodology as that used to determine the colors of the strip's pads. The algorithm determines which color in the color chart is the best match for the color observed in the image. The turbidity measurement algorithm implements the turbidity test used by practitioners, i.e. a text is placed in a legible way in the Cb Compartment such that the sample will be considered cloudy if the text is no longer legible in the presence of a liquid, and different levels of turbidity can be indicated based on the legibility of this text. The algorithm indicates that the urine is cloudy if the text is not visible, or clear if the text is perfectly visible, and other intermediate levels can also be determined. The algorithm also recalculates the level of urine present in the casing by adding the calculated levels in each of the different compartments 188, 189, 190, 191.

With regard to the monitoring of the cat's behavior (time, duration and frequency of passage), the animal is characterized, in addition to a standard profile of a cat, by observing its own habits in the first weeks (behavioral analyses), and values are assigned to the settings for its profile (customized thresholds). If these thresholds are exceeded significantly (i.e. above a predefined tolerance), the owner and veterinarian are automatically notified of any anomalies that could justify a veterinary consultation.

The healthy weight thresholds are set by the veterinarian based on race, age, sex, and following a consultation. If these thresholds are exceeded significantly, the owner and veterinarian are automatically notified of any anomalies that could justify a veterinary consultation.

A urine analysis is proposed to the owner taking into account the animal's profile and the “pathology” thresholdings set by the veterinarian.

In addition to an animal's standard profile, the veterinarian increases the thresholds of 14 analytes making it possible to identify potential pathologies such as diabetes, renal insufficiency, blood problems, etc.

The interpretation of the various anomalies or suspicions makes it possible to ask the owner to make an appointment for a consultation and to keep a sample of the urine collected. The owner therefore brings the casing 175 to this consultation, if it takes place shortly after collection, for example that very day.

Depending on the pathology and its stage of development, a frequency of representative analyses is recommended for a veterinary diagnosis (for example, daily monitoring for diabetes, or monitoring during a treatment or intervention).

Of course, the algorithm can utilize artificial intelligence which, by increasing the data (symptoms, behaviors, results of analyses according to race, age, sex, etc. profiles), will make it possible to predict pathologies and recommend living conditions (food, exercise, etc.) so the animal can live better and longer.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A casing for capturing and analyzing animal urine, that comprises a first urine receptacle and a second urine receptacle which is provided with a support for a strip which carries test specimens comprising reagents,
the receptacles being separated by a partition wall which is provided with a passage, and at least one support that forms a tilting axis between:
a first configuration in which, when the passage is open, the urine flows as a result of gravity into the first receptacle and
a second configuration in which, when the passage is open, the urine flows as a result of gravity into the second receptacle; and
a valve that shuts or opens the passage between said receptacles.

33. The casing of claim 32, wherein the valve is a multiport valve that additionally shuts or opens an inlet for urine in the casing.

34. The casing of claim 33, wherein the valve comprises a holding tank for the first jet of urine entering the casing.

35. The casing of claim 34, wherein the tank holding the first jet of urine is topped by a partition wall limiting the flow of the urine to the second receptacle, in the second configuration of the casing.

36. The casing of claim 32, wherein the first receptacle comprises at least one side compartment and at least one partition wall holding urine in a side compartment when the casing tilts into the second configuration.

37. The casing of claim 32, which comprises a cover, transparent at least opposite the support for a strip, the cover being mobile with respect to a base comprising the receptacles.

38. A device for collecting the urine of a predefined animal, which comprises:
the casing for capturing and analyzing urine of claim 32;
a tray receiving the urinating animal and holding elements inert to the urine, on a screen configured to retain the inert elements and let the urine pass;
a sloping surface, under the screen, for collecting the urine in a convergence area leading to an inlet of the casing for capturing and analyzing urine.

39. The device of claim 8, wherein the means for capturing a first jet of urine comprises a valve on the passage of the urine downstream from the convergence area, the valve being controlled to change position and direct the urine towards a collection area after a certain amount of urine has passed through the valve.

40. The device of claim 38, wherein the casing is at least partially transparent and comprises a tank for collected urine and an access plug for access to the tank by a strip which carries test specimens comprising reagents.

41. The device of claim 38, which further comprises a means for analyzing the residual urine collected by the collection means.

42. The device of claim 41, wherein the analysis means comprises a light sensor for capturing the color of at least one test specimen comprising at least one reagent.

43. The device of claim 42, wherein the analysis means comprises a means for evaluating the color of the urine collected.

44. The device of claim 41, wherein the analysis means comprises a means for evaluating the clarity of the urine collected.

45. The device of claim 41, wherein the analysis means comprises a means for comparing:
at least one evaluated color with at least one color limit value; and/or
at least one evaluated component quantity of the urine with at least one quantity limit value.

46. The device of claim 38, which comprises a base comprising at least one weight sensor.

47. The device of claim 38, which comprises a base comprising a drawer bearing the capture and analysis casing.

48. A method for using a casing according to claim 1, which comprises:
a step of capturing an image of the casing;
a step of identifying a strip in the captured image;
a step of determining the color of pads carried by the strip;
a step of comparing at least one pad color with at least one color limit value, for detecting an anomaly;
a step of identifying at least one compartment holding urine, in the captured image;
a step of measuring the turbidity and/or color of the urine held in at least one compartment; and
a step of comparing the turbidity measured and/or the color measured with at least one limit value for detecting an anomaly; and
a step of displaying each anomaly detected.

49. The method of claim 48, which further comprises:
a step of initializing anomaly detection limit values; and
a step of re-evaluating at least one limit value as a function of values measured for the animal in question.

50. The method of claim 48, which further comprises:
a first step of determining a behavior of the animal with regard to the casing, for example time, duration and frequency of passage;
a step of initializing limit values relative to the animal's behavior;
a second step of values representative of a behavior of the animal; and
a step of comparing measurements with the limit values, for detecting for an anomaly.

51. The method of claim 48, which further comprises:
a first step of determining the weight of the animal and/or the weight of at least one urination of the animal;
a step of initializing limit values relative to the weight determined;
a second step of measuring the weight of the animal and/or the weight of at least one urination of the animal; and
a step of comparing measurements with the limit values, for detecting for an anomaly.
